(19) United States
(12) Patent Application Publication
(10) Pub. No.: US 2024/0299277 A1
(43) Pub. Date: Sep. 12, 2024

(54) COMPOSITION FOR IMPROVING SKIN

(71) Applicant: LG HOUSEHOLD & HEALTH CARE LTD., Seoul (KR)

(72) Inventors: Gwang Jin LEE, Seoul (KR); So Young LEE, Seoul (KR); Mu Hyun JIN, Seoul (KR); Seo Rim MIN, Seoul (KR); Young Bong KIM, Seoul (KR)

(73) Assignee: LG HOUSEHOLD & HEALTH CARE LTD., Seoul (KR)

(21) Appl. No.: 18/269,226

(22) PCT Filed: Dec. 22, 2021
(86) PCT No.: PCT/KR2021/019608

§ 371 Date: Jun. 22, 2023

(30) Foreign Application Priority Data
Dec. 23, 2020 (KR) 10-2020-0182508

(51) Int. Cl. A61K 8/63; A61K 8/34; A61K 8/36; A61K 8/365; A61Q 19/00; A61Q 19/02

(52) U.S. Cl. CPC A61K 8/63; A61K 8/34; A61K 8/361; A61K 8/365; A61Q 19/007; A61Q 19/02; A61K 2800/522; A61K 2800/5922

(57) ABSTRACT

The present invention relates to a skin care composition comprising: cedrol; and at least one selected from the group consisting of beta-sitosterol and linoleic acid; or ferulic acid; and at least one selected from the group consisting of beta-sitosterol and linoleic acid. More specifically, the present invention relates to a cosmetic, a food, and a quasi-drug composition comprising the composition as an active ingredient: improvement of skin whitening, antioxidant, skin elasticity or skin wrinkles, or skin moisturizing. In addition, the composition of the present invention compensates for the low formulation stability of cedrol and ferulic acid and it was found that the effects of antioxidant, melanin reduction, collagen synthesis promotion, and hyaluronic acid synthesis promoter were all synergistically exhibited when cedrol or ferulic acid is used in combination with at least one selected from the group consisting of beta-sitosterol and linoleic acid, compared to when cedrol or ferulic acid is used alone.

### TECHNICAL FIELD

The present invention relates to a skin care composition including: cedrol; and at least one selected from the group consisting of beta-sitosterol and linoleic acid; or ferulic acid; and at least one selected from the group consisting of beta-sitosterol and linoleic acid. More specifically, the present invention relates to a cosmetic, a food, and a quasi-drug composition including the composition as an active ingredient: for skin whitening; for antioxidant; for improvement skin elasticity or skin wrinkles; or improving skin moisturizing.

### BACKGROUND ART

Human skin color is determined by various factors such as melanocyte activity, distribution of blood vessels, thickness of skin, and the presence or absence of pigments inside and outside the body, and in particular, a black pigment called melanin produced by the action of enzymes, such as tyrosinase, in melanocytes is important. Melanin is present in the skin and performs an important function of protecting the body from ultraviolet rays, and the like, but is known to promote pigmentation and skin aging and play a major role in inducing skin cancer when overproduced.

To treat or alleviate excessive melanin pigmentation, previously, ascorbic acid, kojic acid, arbutin, hydroquinone, glutathione, and substances having tyrosinase inhibitory activity have been used in combination with cosmetics or pharmaceuticals. However, their use is limited due to an insufficient whitening effect, safety problems for the skin, stability problems when blended into cosmetics, and the like.

In addition, factors that threaten skin health, such as an increase in an amount of ultraviolet rays due to recent environmental pollution, a westernized lifestyle, and stress, are increasing. The compounds cause skin contamination, dryness, skin trouble, an abnormal skin immune system, and the like, and eventually cause dermatitis, roughness, and rapid skin aging.

In particular, the above compounds increase the concentration of reactive oxygen species in the body, and these reactive oxygen species attack cellular components such as lipids, proteins, sugars, and DNA to cause a peroxidation reaction, thereby accelerating skin aging.

Accordingly, there is a need for the development of a cosmetic composition having an antioxidant effect that suppresses the generation of reactive oxygen species and an anti-aging (prevention of skin aging) effect through this.

Collagen is a major matrix protein produced by fibroblasts of the skin, and performs functions such as maintaining the mechanical firmness of the skin and the bonding strength of tissues, supporting cell adhesion, and inducing cell differentiation. Collagen is reduced by aging and photoaging by ultraviolet irradiation, and collagen reduction is promoted by the activity of the collagenase enzyme that decomposes collagen. It is known that this is closely related to the formation of wrinkles on the skin.

Meanwhile, in the skin, it is known that 50% or more of the hyaluronic acid present in the mammalian body is distributed in the skin, particularly in the intercellular space of the epidermis and the connective tissue of the dermis. It is known that an amount of hyaluronic acid in the skin decreases with aging, and the decrease in hyaluronic acid in the skin is considered to be one of direct causes of the decrease in a moisture content of the skin. Therefore, improving skin moisturizing and hyaluronic acid are closely related.

Currently, retinol, adenosine, chlorella extract, and the like are known as cosmetics for improving skin elasticity and improving skin wrinkles. Retinol is a substance that promotes collagen synthesis and inhibits the elastase enzyme, but is unstable and has limitations in usage due to safety issues such as irritation and rashes when applied to the skin.

In addition, cedrol or ferulic acid is known for its beauty care effect, but due to the inherent properties of the material such as solubility, it is difficult to ensure that the effect is sufficiently delivered through a formulation.

### DISCLOSURE

### Technical Problem

In order to achieve the above object, as a result of intensive research efforts, the present inventors have completed the present invention by ascertaining the following: when cedrol or ferulic acid is combined with at least one selected from the group consisting of beta-sitosterol and linoleic acid, the combination has melanin reduction, collagen synthesis-promoting and hyaluronic acid synthesis-promoting effects, and a combination of cedrol or ferulic acid with at least one selected from the group consisting of beta-sitosterol and linoleic acid may ensure formulation stability compared to the case of preparing formulations with cedrol or ferulic acid alone.

### Technical Solution

In order to achieve the above object, the present invention provides a composition for skin care, including the following as an active ingredient: cedrol, and at least one selected from the group consisting of beta-sitosterol and linoleic acid, for cosmetics, foods, or quasi-drugs.

In addition, the present invention provides a composition for skin care, including the following as an active ingredient: ferulic acid; and at least one selected from the group consisting of beta-sitosterol and linoleic acid, for cosmetics, foods, or quasi-drugs.

In order to achieve the above object, the present invention provides a method for improvement of skin whitening, antioxidant, skin elasticity or skin wrinkles, or skin moisturizing, including: applying, to the skin, a composition including cedrol; and at least one selected from the group consisting of beta-sitosterol and linoleic acid, as an active ingredient.

In order to achieve the above object, the present invention provides a method for improvement of skin whitening, antioxidant, skin elasticity or skin wrinkles, or skin moisturizing, including: administering, to a subject, a composition including cedrol; and at least one selected from the group consisting of beta-sitosterol and linoleic acid, as an active ingredient.

In order to achieve the above object, the present invention provides a use of a composition including cedrol; and at least one selected from the group consisting of beta-sitosterol and linoleic acid, as an active ingredient, for manufacturing cosmetics; foods; or quasi-drugs for skin whitening, antioxidant, improving skin elasticity, improving skin wrinkles, or improving skin moisturizing.

In order to achieve the above object, the present invention provides a method for improvement of skin whitening, antioxidant, skin elasticity or skin wrinkles, or skin moisturizing, including: applying, to the skin, a composition including ferulic acid; and at least one selected from the group consisting of beta-sitosterol and linoleic acid, as an active ingredient.

In order to achieve the above object, the present invention provides a method for improvement of skin whitening, antioxidant, skin elasticity or skin wrinkles, or skin moisturizing, including: administering, to a subject, a composition including ferulic acid; and at least one selected from the group consisting of beta-sitosterol and linoleic acid, as an active ingredient.

In order to achieve the above object, the present invention provides a use of a composition including ferulic acid, and at least one selected from the group consisting of beta-sitosterol and linoleic acid, as an active ingredient, for manufacturing cosmetics; foods, or quasi-drugs for improvement of skin whitening, antioxidant, skin elasticity or skin wrinkles, or skin moisturizing.

### Advantageous Effects

The composition according to the present invention can improve skin with free radical scavenging, melanin reduction, collagen synthesis-promoting and hyaluronic acid synthesis-promoting effects, and since the combination of the present invention has an effect of securing the stability of a formulation, the composition may be effectively used in cosmetics, foods, or quasi-drugs for improving skin.

### BEST MODE FOR IMPLEMENTATION OF THE INVENTION

Hereinafter, the present disclosure will be described in more detail.

As confirmed in experimental examples of the present invention, when a formulation is prepared using only cedrol or ferulic acid, there is a problem in that formulation stability is considerably low (Example 7 and Example 11 of Experimental Example 2). However, in Examples 8 to 10 and Examples 12 to 14 in which cedrol or ferulic acid is combined with at least one selected from the group consisting of beta-sitosterol and linoleic acid, it was confirmed that cedrol or ferulic acid was not precipitated. Therefore, it was confirmed that formulation stability increased when combined with beta-sitosterol and linoleic acid, compared to when cedrol or ferulic acid was used alone.

In addition, it was confirmed that antioxidant, melanin reduction, collagen synthesis-promoting and hyaluronic acid synthesis-promoting effects of cedrol or ferulic acid show a synergistic effect when used in combination with at least one selected from the group consisting of beta-sitosterol and linoleic acid compared to using cedrol or ferulic acid alone.

The present invention provides a cosmetic, food, quasi-drug composition for improvement of skin whitening, antioxidant, skin elasticity or skin wrinkles, or skin moisturizing, including: cedrol; and at least one selected from the group consisting of beta-sitosterol and linoleic acid.

In another aspect, the present invention provides a cosmetic, food, quasi-drug composition for improvement of skin whitening, antioxidant, skin elasticity or skin wrinkles, or skin moisturizing, including: ferulic acid; and at least one selected from the group consisting of beta-sitosterol and linoleic acid.

In another aspect, the present invention provides a method for improvement of skin whitening, antioxidant, skin elasticity or skin wrinkles, or skin moisturizing: applying, to the skin, a composition including cedrol; and at least one selected from the group consisting of beta-sitosterol and linoleic acid, as an active ingredient.

In another aspect, the present invention provides a method for improvement of skin whitening, antioxidant, skin elasticity or skin wrinkles, or skin moisturizing, including: administering, to a subject, a composition including cedrol; and at least one selected from the group consisting of beta-sitosterol and linoleic acid, as an active ingredient.

In another aspect, the present invention provides a use of a composition including cedrol; and at least one selected from the group consisting of beta-sitosterol and linoleic acid, as an active ingredient, for manufacturing cosmetics; foods; or quasi-drugs for improvement of skin whitening, antioxidant, skin elasticity or skin wrinkles, or skin moisturizing.

In another aspect, the present invention provides a method for improvement of skin whitening, antioxidant, skin elasticity or skin wrinkles, or skin moisturizing, including, applying, to the skin, a composition including ferulic acid; and at least one selected from the group consisting of beta-sitosterol and linoleic acid, as an active ingredient.

In another aspect, the present invention provides a method for improvement of skin whitening, antioxidant, skin elasticity or skin wrinkles, or skin moisturizing, including: administering, to a subject, a composition including ferulic acid; and at least one selected from the group consisting of beta-sitosterol and linoleic acid, as an active ingredient.

In another aspect, the present invention provides a use of a composition including ferulic acid; and at least one selected from the group consisting of beta-sitosterol and linoleic acid, as an active ingredient, for manufacturing cosmetics; foods; or quasi-drugs for improvement of skin whitening, antioxidant, skin elasticity or skin wrinkles, or skin moisturizing.

The term “skin whitening” of the present invention refers to all actions that inhibit the synthesis of melanin pigment to brighten skin tone, and suppress or prevent the deposition of melanin in the skin (hyperpigmentation) and alleviate or improve spots and freckles caused by ultraviolet rays, hormones, or heredity.

The term “antioxidant” of the present invention refers to inhibiting the oxidation of cells by free radicals or reactive oxygen species (ROS) having high reactivity according to intracellular metabolism or oxidative stress caused by the influence of ultraviolet rays, and cell damage can be reduced to exhibit anti-aging effects on the skin by removing free radicals or reactive oxygen species. In the present invention, a free radical scavenging effect was confirmed through a DPPH reducing effect confirmed in the experimental examples.

The term “free radical” in the present invention is an atom or molecule having one unpaired electron. A hydrogen atom, or a chlorine atom is a monoatomic free radical, and usually what is called a free radical means that one proton is detached from an inorganic or organic compound molecule and there is one unpaired electron in a residue. Active oxygen has excellent chemical reactivity, and the free radicals induced by active oxygen affect various reactions in the living body and cause aging. Living organisms are always exposed to harmful effects caused by free radicals, and as cells age, harmful effects gradually accumulate to cause various diseases. Thus, free radical scavenging plays an important role in the antioxidant effect.

The term “improvement skin elasticity” of the present invention means alleviating the degree of sagging of the skin. In addition, the term “improvement skin wrinkles” means preventing, suppressing or inhibiting the formation of wrinkles on the skin, or alleviating wrinkles already formed. In the present invention, by confirming a collagen synthesis promoting effect, a improving skin elasticity and improving skin wrinkles effect was confirmed.

The term “skin moisturizing” of the present invention means increasing the feeling of moisture in the skin and maintaining a moist state by suppressing moisture loss. In the present invention, a skin moisturizing effect was confirmed by confirming a hyaluronic acid synthesis promoting effect.

The term “cosmetic composition” of the present invention may be in the form of a general emulsified formulation and a solubilized formulation. For example, the cosmetic composition may have a formulation of cosmetic water such as softening cosmetic water or nourishing cosmetic water, an emulsion such as facial lotion or body lotion, a cream such as a nourishing cream, a moisturizing cream, an eye cream, essence, cosmetic ointment, balm, spray, gel, a pack, sunscreen, a makeup base, a liquid-, solid-, or spray-type foundation, powder, a makeup remover such as a cleansing cream, a cleansing lotion, or a cleansing oil, a cleanser such as cleansing foam, soap, body wash, and the like.

In addition, the cosmetic may contain, in addition to the composition of the present invention, fatty materials, organic solvents, solubilizing agents, thickening agents, gelling agents, softening agents, antioxidants, suspending agents, stabilizers, foaming agents, fragrances, surfactants, water, ionic or nonionic emulsifiers, fillers, sequestering and chelating agents, preservatives, vitamins, blocking agents, wetting agents, essential oils, dyes, pigments, hydrophilic or lipophilic active agents, lipid vesicles, or adjuvants commonly used in cosmetology, such as any other ingredient commonly used in cosmetics.

The cosmetic formulation may include a relatively high concentration of the composition in a wash-off type cosmetic such as a make-up remover, a cleanser, and the like in which active ingredients stay on the skin for a short period of time. On the other hand, in leave-on type cosmetics such as cosmetic water, emulsions, creams, essence, and the like in which active ingredients stay on the skin for a long time, the cosmetics may also include a low concentration of the composition compared to wash-off type cosmetics. Although not limited thereto, in one embodiment of the present invention, the formulation may include 0.0001% by weight to 10% by weight (preferably 0.0001% by weight to 1% by weight) of the composition based on the total weight of the formulation. When the formulation includes less than 0.0001% by weight of the composition of the present invention, sufficient improvement of skin whitening, antioxidant, skin elasticity or skin wrinkles, or skin moisturizing effects cannot be expected, and when it includes more than 10% by weight of the composition, unwanted reactions such as allergies or skin safety problems may occur.

The term “food composition” of the present invention refers to a food composition prepared by adding the above composition to food materials such as beverages, teas, spices, chewing gums, confectioneries, and the like, or encapsulating, powdering, or suspending.

Since the food formulation can be consumed daily, improvement of skin whitening, antioxidant, skin elasticity or skin wrinkles, or skin moisturizing effects can be expected, which is very useful.

When the composition of the present invention is used as a food additive, the composition may be added as it is or used along with other foods or food ingredients, and may be appropriately used according to existing methods. The mixing amount of the active ingredient can be suitably determined according to the purpose of its use (prevention, health or therapeutic treatment). In general, when preparing food or beverage, the composition of the present invention is added in an amount of 15% by parts or less, preferably 10% by parts or less, based on raw materials. However, in long-term intake for the purpose of health and hygiene or health control, the amount may be less than the above range.

There is no particular limitation on the type of food. Examples of foods to which the material may be added include meat, sausage, bread, chocolate, candies, snacks, confectioneries, pizza, ramen, other noodles, gums, ice cream, various soups, beverages, teas, drinks, alcoholic drinks, vitamin complexes, and the like, and include all health foods in a conventional sense.

When the food formulation is a beverage, it may contain various flavoring agents or natural carbohydrates as additional components, like existing beverages. The above-mentioned natural carbohydrates may be monosaccharides such as glucose, fructose; disaccharides such as maltose, sucrose; polysaccharides such as dextrin, cyclodextrin; sugar alcohols such as xylitol, sorbitol, erythritol, etc. As the sweetening agent, natural sweetening agents such as thaumatin, stevia extract; and synthetic sweeteners such as saccharin, aspartame, etc. can be used. The proportion of the natural carbohydrate is generally about 0.01 to 0.04 g, preferably about 0.02 to 0.03 g per 100 mL of the composition of the present invention.

In addition to the above, the food formulation invention may contain various nutrients, vitamins, electrolytes, flavors, colorants, pectic acid and salts thereof, alginic acid and salts thereof, organic acids, protective colloidal thickeners, pH adjusters, stabilizers, preservatives, glycerin, alcohol, carbonation agents used in carbonated beverages, and the like. The food formulation may also contain flesh for the production of natural fruit juices, fruit juice beverages, or vegetable beverages. These components may be used independently or in combination. The proportion of such additives is not critical but is generally selected in the range of 0.01 to 0.1 parts by weight per 100 parts by weight of the composition of the invention.

The term “quasi-drug composition” of the present invention refers to products that are used for the purpose of diagnosing, treating, mitigating, treating, or preventing diseases of humans or animals, but have a milder effect on the human body than pharmaceuticals, and refers to a composition for an article, excluding an apparatus, machine, or device among articles, and an apparatus, machine, or device among articles used for the purpose of pharmacologically affecting the structure and function of humans or animals. Excluding items used for the purpose of pharmaceuticals according to the Pharmaceutical Affairs Act, one embodiment may include, but is not limited to, external preparations for the skin, personal hygiene products, or preparations for internal use, and the formulation method, dosage, usage method, components, and the like of quasi-drugs may be appropriately selected from conventional techniques known in the art.

The present invention provides a pharmaceutical composition for improvement of skin whitening, antioxidant, skin elasticity or skin wrinkles, or skin moisturizing, including: cedrol; and at least one selected from the group consisting of beta-sitosterol and linoleic acid.

In another aspect, the present invention provides a pharmaceutical composition for improvement of skin whitening, antioxidant, skin elasticity or skin wrinkles, or skin moisturizing, including: ferulic acid; and at least one selected from the group consisting of beta-sitosterol and linoleic acid.

The term “pharmaceutical composition” of the present invention may be administered orally or parenterally, and when the composition is used as a pharmaceutical composition, it may be administered in the form of a general pharmaceutical preparation, for example, in various oral and parenteral formulations for clinical administration, and when formulated, it may be prepared using diluents or excipients such as commonly used fillers, extenders, binders, wetting agents, disintegrants, and surfactants, but is not limited thereto.

A solid preparation for oral administration include tablets, pills, powders, granules, capsules, and the like, and such a solid preparation may be prepared by mixing the pharmaceutical composition of the present invention with at least one excipient, for example, starch, calcium carbonate, sucrose or lactose, gelatin, or the like, but is not limited thereto.

In addition to a simple excipient, lubricants such as magnesium stearate and talc are also used. A liquid preparation for oral administration include suspensions, liquids for internal use, emulsions, and syrups, and in addition to water and liquid paraffin, which are commonly used simple diluents, various excipients such as wetting agents, sweeteners, aromatics, and preservatives may be included, but are not limited thereto.

The preparation for parenteral administration includes sterilized aqueous solutions, non-aqueous solutions, suspensions, emulsions, lyophilized preparations, and suppositories. As the non-aqueous solvent and suspending agent, propylene glycol, polyethylene glycol, vegetable oils such as olive oil, injectable esters such as ethyl oleate, etc. can be used. The suppository base may include, but is not limited to, Witepsol, Macrogol, Tween 61, cacao butter, laurin butter, glycerogeratin, and the like.

In addition, the composition of the present invention may be used for preparing the cosmetic formulation. The cosmetic formulations are the same as those previously described in the description of the cosmetic formulation including the composition of the present invention.

Each of the above-mentioned components included in the composition according to the present invention may preferably be included in the compositions of the present invention within a range not exceeding the maximum amount specified in standards prescribed by each government. For example, in the case of providing a pharmaceutical composition, it may be included in the present invention within the scope of the preparation method, properties, usage amount, and the like specified in the pharmacopoeia or pharmaceutical code prescribed in Korea, the United States, Europe, Germany, Japan, China, and the like. In addition, in the case of providing a cosmetic composition, it is included in the cosmetic composition of the present invention within a range that does not exceed the maximum amount specified in the Cosmetics Safety Act prescribed by the government of each country or (Cosmetics Safety Technical Specifications) prescribed by the Chinese government.

Hereinafter, the present invention will be described in detail by way of experimental examples. The following experimental examples merely illustrate the present invention but do not limit the scope of the present invention. In addition, since these experimental examples are only for the purpose of helping the understanding of the present invention, the scope of the present invention is not limited thereto in any sense.

### Experimental Example 1: Confirmation of Skin Care Effect

TABLE 1

| Sample number | Composition |
|---|---|
| Comparative Example 1 | Cedrol |
| Comparative Example 2 | Ferulic acid |
| Comparative Example 3 | Beta-sitosterol |
| Comparative Example 4 | Linoleic acid |
| Example 1 | Cedrol + Beta-sitosterol |
| Example 2 | Cedrol + Linoleic acid |
| Example 3 | Cedrol + Beta-sitosterol + Linoleic acid |
| Example 4 | Ferulic acid + Beta-sitosterol |
| Example 5 | Ferulic acid + Linoleic acid |
| Example 6 | Ferulic acid + Beta-sitosterol + Linoleic acid |

Samples were prepared by dissolving the components in methanol as described above. A concentration of each component of the sample was adjusted according to the experimental methods of Experimental Examples 1 to 4 below.

### <Experimental Example 1-1> Antioxidant Effect According to Free Radical Scavenging

In Comparative Examples 1 to 4 and Examples 1 to 6, a solution was prepared using methanol as a solvent so that the concentration of each component in the solution was 0.5 mg/mL. In order to confirm the antioxidant effect of Comparative Examples 1 to 4 and Examples 1 to 6, free radical scavenging ability was measured using a 1,1-diphenyl-2-picrylhydrazyl (DPPH) method. DPPH is a relatively stable free radical and exhibits a maximum absorption at 517 nm when present in a radical state, but loses its absorption ability when the radical is scavenged. DPPH was used after being dissolved in methanol at a concentration of 0.12 mM.

Comparative Examples 1 to 4, Examples 1 to 6, and Trolox solutions by concentration (0.0125, 0.025, 0.05, 0.1, 0.2, 0.4, 0.8 mg/mL) were each put into a 24-well plate in an amount of 100 μL, and the DPPH solution was added in an amount of 1,900 μL. After blocking the light at room temperature and leaving it for 1 hour, the absorbance at 517 nm was measured using a microplate reader (BioTek EPOCH). A calibration curve according to the concentration was created using the absorbance values of the Trolox solution at 7 concentrations. In this experiment, Trolox is one indicator for measuring the antioxidative capacity of a sample, and the antioxidative capacity of each comparative example and example is a size compared to Trolox. The degree of antioxidant effect shown by 1 mL of the example solution by substituting the absorbance of the example solution into the calibration curve is expressed in Trolox μmol equivalents/mL and summarized in Table 2 below. The experiment was performed three times, and then an average value was calculated and shown.

TABLE 2

| | Comp. | Comp. | Comp. | Comp. | | | | | | |
|---|---|---|---|---|---|---|---|---|---|---|
| Sample | Ex. 1 | Ex. 2 | Ex. 3 | Ex. 4 | Ex. 1 | Ex. 2 | Ex. 3 | Ex. 4 | Ex. 5 | Ex. 6 |
| Trolox μmol | 0.104 | 0.981 | 0.051 | 0.023 | 0.163 | 0.148 | 0.213 | 1.051 | 0.998 | 1.026 |
| equivalents/mL | | | | | | | | | | |

Comp. Ex.: Comparative Example

### Ex.: Example

As shown in Table 2, it was confirmed that all of the examples of the present invention had sufficient radical scavenging ability.

### <Experimental Example 1-2> Whitening Effect According to Inhibition of Melanin Production

To culture MNT-1 melanoma cells, a mixture of Dulbecco's Modified Eagle's Medium (DMEM) and 10% fetal bovine serum (FBS) was used as a basal medium. MNT-1 melanoma cells were dispensed in a 6-well plate at a concentration of 1-2×10⁵ cells/mL and cultured for 24 hours. Samples were treated so that the final concentration of each component of Comparative Examples 1 to 4 and Examples 1 to 6 was 3 μg/mL (3 ppm), and then cultured for 72 hours. As a positive control, arbutin was treated to be 200 μg/mL (200 ppm) (positive control in Table 3). Thereafter, the cells were treated with trypsin, detached from the culture vessel, centrifuged at 13,000 rpm for 1 minute, and then the supernatant was removed. Cells were lysed by adding 300 μL of a 0.5% Triton X-100 solution to the remaining cells. This was again centrifuged at 13,000 rpm for 3 minutes to recover the pellet and supernatant separately. Melanin in the pellet was dissolved by adding 100 μL of a 0.5 N sodium hydroxide solution and incubating for 12 hours, and absorbance at 450 nm was measured using a spectrophotometer to measure the total amount of melanin produced.

The total amount of inhibited melanin production was calculated as a melanin production inhibition rate (%) relative to a group (negative control group) treated with methanol (Equation 1 below), and the experiment was performed three times, and then an average value was calculated and shown in Table 3.

$$Melanin production inhibition rate ( \% ) = ( 1 - ( total amount of melanin produced in group treated with each sample / total amount of melanin produced in group treated with methanol ) ) * 100 \qquad ( Equation 1 )$$

TABLE 3

| | Positive | Comp. | Comp. | Comp. | Comp. | | | | | | |
|---|---|---|---|---|---|---|---|---|---|---|---|
| Sample | Control | Ex. 1 | Ex. 2 | Ex. 3 | Ex. 4 | Ex. 1 | Ex. 2 | Ex. 3 | Ex. 4 | Ex. 5 | Ex. 6 |
| Melanin | 19.8% | 17.5% | 16.3% | 1.7% | 6.7% | 18.1% | 19.0% | 20.1% | 15.4% | 19.9% | 18.5% |
| production | | | | | | | | | | | |
| Inhibition | | | | | | | | | | | |
| rate (%) | | | | | | | | | | | |

Comp. Ex.: Comparative Example
Ex.: Example

As shown in Table 3, it was confirmed that all of the examples of the present invention have sufficient melanin production inhibitory effects.

### <Experimental Example 1-3> Collagen Synthesis Promoting Effect

A mixture of DMEM and 10% FBS was used as a basal medium for culturing human-derived fibroblasts. Skin fibroblasts were dispensed in a 48-well plate at a concentration of 2-5×10⁴ cells/mL and cultured for 24 hours. The basal medium with growth factors was removed, and a medium (a medium to which FBS was not added) having a final concentration of 3 μg/mL (3 ppm) of each component of Comparative Examples 1 to 4 and Examples 1 to 6 was added and cultured for 24 hours. A positive control for inducing collagen synthesis was treated with TGF-β to be 5 ng/mL (5 ppb) (positive control in Table 4). A cell culture solution was taken and synthesized collagen was measured using a Human Procollagen 1α1 Duoset ELISA Kit (R&D Systems) and a spectrophotometer.

A calibration curve was created by measuring the separated and purified collagen at concentrations of 0.25, 0.5, 1, 2, 4, and 8 ng/mL, and based on this, the collagen (Type I collagen) production amounts of Comparative Examples 1 to 4 and Examples 1 to 6, a negative control group, and a positive control group were calculated. The collagen production amount was calculated using a collagen production rate (%) relative to the group treated with methanol (negative control group) (Equation 2 below), and the experiment was performed three times, and then an average value was calculated and shown in Table 4.

$$Collagen production rate ( \% ) = ( collagen production amount of group treated with each example - collagen production amount of group treated with methanol ) / ( collagen production amount of group treated with methanol ) * 100 \qquad ( Equation 2 )$$

TABLE 4

| | Positive | Comp. | Comp. | Comp. | Comp. | | | | | | |
|---|---|---|---|---|---|---|---|---|---|---|---|
| Sample | Control | Ex. 1 | Ex. 2 | Ex. 3 | Ex. 4 | Ex. 1 | Ex. 2 | Ex. 3 | Ex. 4 | Ex. 5 | Ex. 6 |
| Collagen | 32.1% | 27.9% | 8.8% | 2.4% | −2.5% | 27.6% | 21.2% | 23.5% | 6.6% | 7.6% | 4.1% |
| Production rate | | | | | | | | | | | |
| (%) | | | | | | | | | | | |

Comp. Ex.: Comparative Example
Ex.: Example

As shown in Table 4, a sufficient collagen enhancing effect was confirmed in all examples of the present invention.

### <Experimental Example 1-4> Hyaluronic Acid Synthesis-Promoting Effect

Human skin keratinocytes (primary cultured human skin keratinocytes) were purchased from Thermo Fisher Scientific and cultured using Keratinocyte-SFM supplemented with epidermal growth factor (EGF), which is a growth factor, and bovine pituitary extract (BPE). The keratinocytes were dispensed in a 60-mm culture plate at a concentration of 2-5×10⁵ cells/mL and cultured for 24 hours. The basal medium with growth factors was removed, and a medium (a medium to which EGF and BPE were not added) having a final concentration of 3 μg/mL (3 ppm) of each component of Comparative Examples 1 to 4 and Examples 1 to 6 was added and cultured for 24 hours. A positive control for inducing hyaluronic acid synthesis was treated with all-trans-retinoic acid to be 100 nM (positive control in Table 5). A cell culture solution was taken and synthesized hyaluronic acid was measured using a Hyaluronan DuoSet ELISA Kit (R&D Systems) and a spectrophotometer.

A calibration curve was created by measuring standard hyaluronic acid at concentrations of 1.1, 3.3, 10, 30, and 90 ng/mL, and based on this, the hyaluronic acid production amounts of Comparative Examples 1 to 4 and Examples 1 to 6, a negative control group, and Comparative Example 3 were calculated. The hyaluronic acid production amount was calculated using a hyaluronic acid production rate (%) relative to the group treated with methanol (negative control group) (Equation 2 below), and the experiment was performed three times, and then an average value was calculated and shown in Table 5.

$$Hyaluronic acid production rate ( \% ) = ( hyaluronic acid production amount of group treated with example - hyaluronic acid production amount of a group treated with methanol ) / ( hyaluronic acid production amount of group treated with methanol ) * 100 \qquad ( Equation 3 )$$

TABLE 5

| | Positive | Comp. | Comp. | Comp. | Comp. | | | | | | |
|---|---|---|---|---|---|---|---|---|---|---|---|
| Sample | Control | Ex. 1 | Ex. 2 | Ex. 3 | Ex. 4 | Ex. 1 | Ex. 2 | Ex. 3 | Ex. 4 | Ex. 5 | Ex. 6 |
| Hyaluronic | 45.9% | 5.6% | 32.2% | −3.0% | 4.1% | 3.9% | 4.4% | 2.8% | 36.3% | 42.2% | 42.9 |
| acid | | | | | | | | | | | |
| Production | | | | | | | | | | | |
| rate (%) | | | | | | | | | | | |

Comp. Ex.: Comparative Example
Ex.: Example

As shown in Table 5, it was confirmed that all of the examples of the present invention had sufficient hyaluronic acid production effects.

### Experimental Example 2: Confirmation of Formulation Stability Improvement

### <Experimental Example 2-1> Preparation of Cosmetic Composition Containing Cedrol (Examples 7 to 10)

In Example 7, in a composition ratio (units:weight %) shown in Table 6 below, raw materials 3 and 4 were dispersed in raw material 2, and then raw material 5 was added and dissolved. After confirming the dissolution, raw materials 6-8 were dissolved and then stirred uniformly. Raw materials 9-11 were sequentially added to raw material 1 and stirred uniformly, and an ethanol phase was gradually added to an aqueous phase and stirred uniformly for 10 minutes to prepare a cosmetic solution. Examples 8-10 were prepared in the same manner as Example 7.

TABLE 6

| Raw material | Component name | Example 7 | Example 8 | Example 9 | Example 10 |
|---|---|---|---|---|---|
| 1 | Purified water | To 100 | To 100 | To 100 | To 100 |
| 2 | Ethanol | 2 | 2 | 2 | 2 |
| 3 | Beta-sitosterol | — | 0.1 | — | 0.1 |
| 4 | Linoleic acid | — | — | 0.1 | 0.1 |
| 5 | Cedrol | 0.05 | 0.05 | 0.05 | 0.05 |
| 6 | Solubilizer | 1 | 1 | 1 | 1 |
| 7 | Fragrance | Appropriate | Appropriate | Appropriate | Appropriate |
| | | amount | amount | amount | amount |
| 8 | Hydrogenated | 0.3 | 0.3 | 0.3 | 0.3 |
| | polydecene | | | | |
| 9 | Carbomer | 0.15 | 0.15 | 0.15 | 0.15 |
| 10 | Neutralizer | Appropriate | Appropriate | Appropriate | Appropriate |
| | | amount | amount | amount | amount |
| 11 | Antiseptic | Appropriate | Appropriate | Appropriate | Appropriate |
| | | amount | amount | amount | amount |

### <Experimental Example 2-2> Preparation of Cosmetic Composition Containing Ferulic Acid (Examples 11 to 14)

In Example 11, in a composition ratio (units:weight %) shown in Table 7 below, raw materials 3 and 4 were dispersed in raw material 2, and then raw material 5 was added and dissolved. After confirming the dissolution, raw materials 6-8 were dissolved and then stirred uniformly. Raw materials 9-11 were sequentially added to raw material 1 and stirred uniformly, and an ethanol phase was gradually added to an aqueous phase and stirred uniformly for 10 minutes to prepare a cosmetic solution. Examples 12-14 were prepared in the same manner as Example 11.

TABLE 7

| Raw | | | | | |
|---|---|---|---|---|---|
| material | Component name | Example 11 | Example 12 | Example 13 | Example 14 |
| 1 | Purified water | To 100 | To 100 | To 100 | To 100 |
| 2 | Ethanol | 2 | 2 | 2 | 2 |
| 3 | Beta-sitosterol | — | 0.2 | — | 0.2 |
| 4 | Linoleic acid | — | — | 0.2 | 0.2 |
| 5 | Ferulic acid | 0.1 | 0.1 | 0.1 | 0.1 |
| 6 | Solubilizer | 1 | 1 | 1 | 1 |
| 7 | Fragrance | Appropriate | Appropriate | Appropriate | Appropriate |
| | | amount | amount | amount | amount |
| 8 | Hydrogenated | 0.3 | 0.3 | 0.3 | 0.3 |
| | polydecene | | | | |
| 9 | Carbomer | 0.15 | 0.15 | 0.15 | 0.15 |
| 10 | Neutralizer | Appropriate | Appropriate | Appropriate | Appropriate |
| | | amount | amount | amount | amount |
| 11 | Antiseptic | Appropriate | Appropriate | Appropriate | Appropriate |
| | | amount | amount | amount | amount |

### <Experimental Example 2-3> Evaluation of Solubility and Compatibility

Solubility and compatibility were evaluated for the cosmetic compositions of Examples 7 to 14. While stored for 4 weeks at 25 C, 4 weeks at 4° C., and 4 weeks at 45° C., phase separation and precipitation were visually observed and shown in Tables 8 and 9 below.

### <Evaluation Criteria>

No abnormality: ◯, Weak precipitation: Δ, Separation and precipitation: X

TABLE 8

| | Example 7 | Example 8 | Example 9 | Example 10 |
|---|---|---|---|---|
| Room | X | Δ | ◯ | ◯ |
| temperature | | | | |
| (25° C.) | | | | |
| Refrigerated | Δ | ◯ | ◯ | ◯ |
| (4° C.) | | | | |
| High | X | ◯ | ◯ | ◯ |
| temperature | | | | |
| (45° C.) | | | | |

As shown in Table 8, in Examples 8 to 10 using beta-sitosterol and linoleic acid, it was confirmed that the cedrol was dissolved in water. In Example 7 in which the beta-sitosterol and linoleic acid were not used, it was confirmed that the cedrol was dispersed as it was in powder form and did not dissolve.

TABLE 9

| | Example 11 | Example 12 | Example 13 | Example 14 |
|---|---|---|---|---|
| Room | X | ◯ | ◯ | ◯ |
| temperature | | | | |
| (25° C.) | | | | |
| Refrigerated | X | Δ | Δ | ◯ |
| (4° C.) | | | | |
| High | X | ◯ | ◯ | ◯ |
| temperature | | | | |
| (45° C.) | | | | |

As shown in Table 9, in Examples 12 to 14 using beta-sitosterol and linoleic acid, it was confirmed that ferulic acid was dissolved in water. In Example 14, in which beta-sitosterol and linoleic acid were simultaneously used, it was confirmed that a stable formulation was maintained without precipitation even under refrigerated conditions. In Example 11 in which the beta-sitosterol and linoleic acid were not used, it was confirmed that the ferulic acid was dispersed as it was in powder form and did not dissolve.

Through the above experimental results, it was confirmed that beta-sitosterol and linoleic acid of the present invention affect the solubility of cedrol and ferulic acid, and thus help cedrol and ferulic acid to be stably dispersed in cosmetics.

In addition, it was confirmed that each of cedrol and ferulic acid of the present invention can be stably dispersed in cosmetics when treated along with beta-sitosterol and linoleic acid.

1. A composition, comprising:
cedrol or ferulic acid; and
one or more selected from the group consisting of beta-sitosterol and linoleic acid.

2. The composition of claim 1, wherein the composition is cosmetic, food, or quasi-drug composition.

3. The composition of claim 1, wherein the composition has a melanin reducing effect.

4. (canceled)

5. (canceled)

6. The composition of claim 1, wherein the composition has a free radical scavenging effect.

7. (canceled)

8. (canceled)

9. The composition of claim 1, wherein the composition has a collagen synthesis promoting effect.

10. (canceled)

11. (canceled)

12. The cosmetic composition of claim 1, wherein the composition has a hyaluronic acid synthesis promoting effect.

13. (canceled)

14. The composition of claim 1, wherein the composition is used for the composition for improving skin,
wherein the improvement of skin is one or more selected from the group consisting of improvement of skin whitening, antioxidant, improvement of skin elasticity or skin wrinkles, and skin moisturizing.

15. (canceled)

16. (canceled)

17. A method for improving skin, comprising:
applying to the skin or administering to a subject, a composition including cedrol or ferulic acid; and at least one selected from the group consisting of beta-sitosterol and linoleic acid, as an active ingredient, wherein the improvement of skin is one or more selected from the group consisting of improvement of skin whitening, antioxidant, improvement of skin elasticity or skin wrinkles, and skin moisturizing.

18. The method of claim 17, wherein the composition is cosmetic, food, or quasi-drug composition.

19. The method of claim 17, wherein the composition has a melanin reducing effect.

20. The method of claim 17, wherein the composition has a free radical scavenging effect.

21. The method of claim 17, wherein the composition has a collagen synthesis promoting effect.

22. The method of claim 17, wherein the composition has a hyaluronic acid synthesis promoting effect.
